(19) United States
(12) Patent Application Publication
(10) Pub. No.: US 2005/0019844 A1
(43) Pub. Date: Jan. 27, 2005

(54) Method of detecting malignant tumor and detection kit

(76) Inventor: Katsu Honzawa, Shizuoka (JP)

Correspondence Address: MORGAN LEWIS & BOCKIUS LLP, 1111 PENNSYLVANIA AVENUE NW, WASHINGTON, DC, 20004, US

(21) Appl. No.: 10/495,876

(22) PCT Filed: Nov. 20, 2002
(86) PCT No.: PCT/JP02/12133

(30) Foreign Application Priority Data
Nov. 22, 2001 (JP) 2001-358051

(51) Int. Cl. G01N 33/574

(52) U.S. Cl. 435/7.23

(57) ABSTRACT

A malignant tumor detection method suitable for the primary screening for a malignant tumor, and a detection kit using the method are provided. Considering various amino group-containing low molecular weight tumor markers in a sample, a tumor marker is labeled under suitable conditions using an amino group labeling dye which reacts specifically with it. The labeled tumor marker is subjected to spectroscopic measurement, and the tumor marker amount present in the sample is calculated. The presence or absence of a malignant tumor is detected by comparing the tumor marker amount with a threshold value for the tumor marker obtained from healthy persons. The series of these steps is performed using the detection kit.

### TECHNICAL FIELD

This invention relates to a malignant tumor detection method and detection kit, and in particular, relates to a simple malignant tumor detection method and detection kit using an amino group-containing low molecular weight tumor marker, in order to discover a malignant tumor at an early stage.

### BACKGROUND ART

Examples of low molecular weight tumor markers for malignant tumors known in the art include amino group-containing compounds, such as 3-hydroxyproline, pteridines and polyamine. Clinical research on such amino group-containing compounds began to attract attention, after Russell et al found in 1971 that polyamine excretion increased in cancer patient urine (Russel, D. H. et al: Cancer Res., 31, 1555-1558 (1971)).

A widely used method for analyzing polyamine is the enzymatic method. In this method, the total amount of free polyamine (cadaverine, spermine, putrescine) is measured using acylpolyamine amidohydrolase. Using this method, many kinds of cancer, such as esophageal carcinoma, gastric cancer, colon cancer, cholangiocarcinoma, pancreatic cancer, lung cancer, ovarian cancer and prostatic cancer can be detected with a sensitivity of close to 50% (Akiyuki Okubo, Medical Clinics of Japan, 24, 11, 2234-2238 (1998)).

3-hydroxyproline is an amino acid specific to Type IV collagen which is a component of basal membrane. If cancer cells proliferate and basal membrane is broken, 3-hydroxyproline is excreted in urine (Kubochi, K. et al: Development of a direct measurement assay for collagenase against Type I and Type IV collagens in tissue homogenate and its application in stomach and lung cancers. In Proteinases in Inflammation and Tumor Invasion (Tschesche, H. ed.), 337-356, Walter de Gruyter and Co., Berline, 1986). Okazaki et al measured the 3-hydroxyproline amount in the urine of 97 patients suffering from various types of cancer and 211 healthy persons by the ninhydrin method using an automatic amino acid analyzer. As a result, the sensitivity of 3-hydroxyproline was 42%. On the other hand, the percentage of healthy persons showing an abnormal value was only 2% (Okazaki, I. et al.: J. Lab. Clin. Med., 120, 908-920 (1992)).

To detect and assay the various aforesaid amino group-containing low molecular weight tumor markers, liquid column chromatography has been used conventionally. In liquid column chromatography, it takes long time to establish the analysis conditions suitable for a specific marker. Further, separation procedure and detection also take long time, and the detection reproducibility is not good. The chromatography unit itself also has the disadvantage of being difficult to miniaturize. Hence, its clinical application to the diagnosis of a test subject was very difficult.

### DISCLOSURE OF THE INVENTION

It is therefore an object of this invention, which was conceived in view of the present situation, to provide a simple and rapid malignant tumor detection method and detection kit using an amino group-containing low molecular weight tumor marker.

In order to attain the aforesaid object, the Inventor considered it a pressing need to establish a method of simply detecting and assaying an amino group-containing low molecular weight tumor marker without depending on liquid chromatography.

It was thus discovered that the amino group-containing low molecular weight tumor marker in a sample could be selectively labeled (even in the presence of other amino group-containing low molecular weight compounds in vivo) by an amino acid labeling dye, and that the amount of this labeled amino group-containing low molecular weight tumor marker could be assayed by a spectroscopic method. Further, by comparing the value of the amino group-containing low molecular weight tumor marker amount in the sample calculated in this way with a threshold for this tumor marker, a malignant tumor could be detected simply and rapidly, and this led to the present invention.

Specifically, the present invention provides a malignant tumor detection method using an amino group-containing low molecular weight tumor marker in a sample, comprising the steps of:
(a) reacting the sample with an amino group labeling dye to produce a labeled amino group-containing low molecular weight tumor marker;
(b) measuring the fluorescence or absorbance of the sample after the step (a);
(c) calculating the amount of the amino group-containing low molecular weight tumor marker in the sample from the measured value obtained in the step (b); and
(d) comparing the calculated value obtained in the step (c) with the threshold value for the amino group-containing low molecular weight tumor marker, where the result is deemed to be “positive” for the presence of a malignant tumor if the calculated value is larger than the threshold.

Unlike tumor markers such as antigens and antibodies, the low molecular weight tumor marker is nonspecific for tumor tissues, and the detection method of this invention is suitable for the primary screening for a malignant tumor.

In the aforesaid malignant tumor detection method, a step of pretreating the sample prior to the step (a) may be included.

In the aforesaid malignant tumor detection method, a step of performing a concentration correction of the calculated value of the amino group-containing low molecular weight tumor marker may further be included between the steps (c) and (d).

In the aforesaid malignant tumor detection method, the amino group labeling dye is preferably 4-chloro-7-nitrobenzofurazan or 4-fluoro-7-nitrobenzofurazan.

In the aforesaid malignant tumor detection method, the amino group labeling dye is preferably immobilized on a solid phase in advance.

In the aforesaid malignant tumor detection method, the step (b) is preferably performed by fluorometry.

In the aforesaid malignant tumor detection method, the amino group-containing low molecular weight tumor marker is preferably 3-hydroxyproline.

In the aforesaid malignant tumor detection method, the sample is preferably urine.

In the aforesaid malignant tumor detection method, the concentration correction is preferably performed by the creatinine amount in the sample.

The present invention further provides a malignant tumor detection kit using an amino group-containing low molecular weight tumor marker in a sample, comprising at least a standard solution of the amino group-containing low molecular weight tumor marker, an amino group labeling dye and a buffer.

In the aforesaid malignant tumor detection kit, the amino group labeling dye is preferably immobilized on a solid phase.

### BRIEF DESCRIPTION OF THE DRAWINGS

FIG. 1 is a diagram showing measured values of the fluorescence intensity of the reaction product of 3-hydroxyproline and 4-chloro-7-nitrobenzofurazan (NBD-Cl) in urine specimens sampled from healthy persons and cancer patients according to the method of the present invention, wherein the measured values corrected by the creatine concentration in the urine specimen for each sample have been plotted for the healthy person group and the malignant tumor patient group.

### BEST MODE FOR CARRYING OUT THE INVENTION

The malignant tumor detection method and detection kit according to the present invention will now be described in detail.

According to the invention, a malignant tumor is detected by selectively labeling an amino group-containing low molecular weight tumor marker in the sample from a test subject, and assaying the labeled amino group-containing low molecular weight tumor marker.

(Amino Group-containing Low Molecular Weight Tumor Marker)

Tumor markers are substances which tumor cells produce in large amounts, and they are normally undetected substances which are also referred to as tumor-related antigens. Autoantibodies to these antigens are likewise generated only after the antigen is produced, which is why it can be used as a tumor marker. In addition, tumerigenic cells produce large amounts of hormones, enzymes and specific low molecular weight compounds, all of which may be useful to detect the presence of a malignant tumor (cancer).

The present invention utilizes an amino group-containing low molecular weight tumor marker, in particular, 3-hydroxyproline as a tumor marker. The reason for using an amino group-containing low molecular weight tumor marker, as was already mentioned, is that it permits easier detection and assay than a macromolecule such as an antigen or antibody, and is suitable for the nonspecific detection of malignant tumors (tissues or organs) (i.e., primary screening).

(Amino Group Labeling Dye)

The present invention utilizes an amino group labeling dye for labeling an amino group-containing low molecular weight tumor marker, and when the marker is 3-hydroxyproline, 4-chloro-7-nitrobenzofurazan (NBD-Cl) or 4-fluoro-7-nitrobenzofurazan (NBD-F) is preferably used as the amino group labeling dye, with NBD-Cl being more preferred.

(Sample)

The sample used in the invention may be any biological specimen usually sampled in in vitro diagnosis, such as serum, plasma, urine, spinal fluid, amniotic fluid, ascites or lymphocytes, and rinses or extracts obtained by washing tissue or cells with solvents. Among these, urine and serum are preferable, and urine is particularly preferred from the viewpoint that it can be sampled without pain unlike collections of blood.

(Pretreatment of Sample)

In order to react the sample with the amino group labeling dye, and label the amino group-containing low molecular weight tumor marker contained in the sample, it may be preferable to pretreat the sample before the labeling reaction. Although the pretreatment method used by the invention varies with the sample type, a known method may be used for a specific sample. For example, if the sample is urine, the urine sampled from the test subject is centrifuged, and the supernatant is hydrolyzed by reaction with hydrochloric acid. Subsequently, the hydrolyzed urine supernatant is lyophilized, and reconstituted in a buffer. The buffer used for the reconstitution may be borate buffer, phosphate buffer or Tris buffer.

If necessary, the urine sample may be diluted with an organic solvent miscible with water. Examples of an organic solvent which may be used for this purpose are dimethylsulfoxide (DMSO), dimethylformamide (DMF) and ethanol. If the concentration of the labeled amino group-containing low molecular weight tumor marker in the sample is too low for spectroscopy measurement (described later), it may be concentrated to an appropriate concentration. Specifically, the sample may be concentrated by removal of solvent, chromatography or the like.

(Labeling Reaction of Amino Group-containing Low Molecular Weight Tumor Marker)

Subsequently, the sample is allowed to react with the amino group labeling dye, and the amino group-containing low molecular weight tumor marker in the sample is thereby labeled. In a particularly preferred form of the invention, if 3-hydroxyproline is selected as the amino group-containing low molecular weight tumor marker and NBD-Cl is selected as the amino group labeling dye, a classical reaction is carried out as follows. The sample is brought in contact with NBD-Cl, and incubated at room temperature (about 20-50° C.) for a predetermined time (1 to 2 minutes). This reaction time is a sufficient time only for NBD-Cl and 3-hydroxyproline to react, and even in the presence of amino group-containing low molecular weight compounds (contaminant amino acids, etc.) in the sample, as these have a low reactivity with NBD-Cl under the aforesaid reaction conditions, almost no reaction products which might interfere with the spectroscopic measurement are generated.

The catalytic reaction can be carried out if the reaction system is a liquid phase, or a liquid phase/solid phase system. In the former case, NBD-Cl is insoluble in water, and therefore is used in solution in an organic solvent which is miscible with water. Specific examples of such an organic solvent are methanol, ethanol, diethyl ether, ethyl acetate, DMF, DMSO, acetone and acetonitrile. Among these, acetonitrile is particularly preferred.

The pH of the reaction system is preferably kept constant (particularly, within the range of approx. 8.0 to 9.0). For this purpose, a buffer is added to the reaction system. Suitable buffers are borate buffer, phosphate buffer and Tris buffer.

(Immobilization)

When inspecting many samples at once using the malignant tumor detection method of the invention, it is preferred to carry out the labeling reaction in a liquid phase/solid phase system.

For such purpose, the amino group labeling dye to be used is immobilized on a solid phase. Examples of a suitable solid phase are a tube (glass or plastic) containing a microtube, a plate (for example, a 96/384 micro well plate), a pipette tip, polystyrene beads, latex particles or magnetic particles. The material thereof and its shape are not particularly limited provided that is a material which does not interfere with the spectroscopy measurement of the sample after the labeling reaction. The immobilization method used may be physical adsorption, covalent bond or any other method known in the art. In the case that the amino group labeling dye is immobilized on the solid phase by physical adsorption, a surfactant and/or a dispersion adjuvant are preferably used to disperse and immobilize the amino group labeling dye more uniformly. Examples of a suitable dispersion adjuvant are polyethylene glycol, cyclodextrin and dextran. Examples of a suitable surfactant are Triton-X, sodium dodecyl sulfate (SDS) and Tween-20.

(Spectroscopic Measurement)

In the next step, the amino group-containing low molecular weight tumor marker labeled with the amino group labeling dye is measured spectroscopically, and the amino group-containing low molecular weight tumor marker amount present in the sample is calculated. There is no particular limitation on the spectroscopic method provided it can measure the fluorescence or absorbance of the labeling dye.

If 3-hydroxyproline is the low molecular weight tumor marker and NBD-Cl is the amino group labeling dye, the labeled amino compound has a fluorescence peak in the vicinity of about 550 nm, and an absorption peak in the vicinity of about 500 nm. Therefore, in this case, an apparatus which can measure the absorption spectrum of the visible region in the range of about 350 to 600 nm is desirable. An apparatus which allows the use of an excitation wavelength of about 250 to 550 nm, and which can measure the fluorescence spectrum in the range of about 350 to 600 nm, is also desired.

When measuring the 3-hydroxyproline amount, it is more preferred from the viewpoint of detection sensitivity to measure the fluorescence spectrum (fluorescence intensity) of a fluorescent reaction product with NBD-Cl.

A calibration curve of the amino compound is constructed in advance by reacting a authentic sample of the amino compound, the amino group-containing low molecular weight tumor marker, with the amino-group labeling dye, measuring the value of the fluorescence intensity or absorbance of the labeled amino compound, and plotting this against the concentration of the amino compound used. The measured value of the fluorescence intensity or absorbance obtained from the sample is then compared with this calibration curve, and the labeled amino group-containing low molecular weight tumor marker amount in the sample is calculated.

(Concentration Correction)

When the sample is urine, it is necessary to make a concentration correction of the calculated value of the amino group-containing low molecular weight tumor marker amount obtained above. This is because a test subject's water intake and water divergence differ from each other, and a dilution error rises in the calculated value. For this purpose, the creatine concentration in the sample is assayed, and the calculated value is corrected based thereon.

(Detection of Malignant Tumor)

The malignant tumor is detected on the basis of the amino group-containing low molecular weight tumor marker amount in the sample which was calculated as described above. Herein, the amino group-containing low molecular weight tumor marker amount in the sample of healthy persons is calculated in the same way as a comparison. Here also, if the aforesaid concentration correction is necessary, it is performed in a similar manner and a threshold for the amino group-containing low molecular weight tumor marker is obtained. This threshold is compared with the calculated value (or corrected value) of the amino group-containing low molecular weight tumor marker in the test subject's sample. As a result, if the calculated value is larger than this threshold, the test subject is deemed to be “positive” regarding the presence of a malignant tumor, and a malignant tumor can thus be detected. It should be stressed that the intention of the invention is to provide a means for primary screening of a malignant tumor. A positive result means only that the test subject may suffer from a malignant tumor.

Preferably, a test subject who received such a positive result will proceed to inspections using other organ-specific diagnostic drugs (such as CEA, AFP, PSA) or diagnostic methods, a biopsy, X-ray, MRI and the like.

(Detectable Malignant Tumors)

The primary or metastatic malignant tumors which can be diagnosed using the detection method and detection kit of the invention are very varied, representative examples being breast cancer, prostatic cancer, liver cancer, lung cancer, colorectal cancer, gastric cancer, pancreatic cancer, bladder cancer, head/neck cancer, kidney cancer, cervical cancer, uterine cancer, thyroid cancer, brain tumors, tongue cancer, lymphoma, multiple myeloma, melanoma and leukemia. By using the detection method and detection kit of the invention, these malignant tumors can be detected at various stages of progression, in particular at the first stage, and a discrimination can be made from the presence of a benign tumor or the absence of a malignant tumor in the subject being screened.

(Detection Kit)

In everyday diagnosis, a kit which does not require complicated operation is preferred so that a malignant tumor can be detected easily. As many physiologically active substances are present in vivo, a detection kit is required which can specifically discriminate and assay tumor markers. The invention provides a detection kit suitable for the malignant tumor detection method of the invention which satisfies these various requirements, and comprises at least a standard solution of an amino group-containing low molecular weight tumor marker, an amino group labeling dye and a buffer. In addition to these components, it may also contain a sample diluent (solvent), solubilizing agent, solid phase conversion standard reagent and positive control.

The buffer may be provided in the form of a solid or a liquid, such as borate buffer, phosphate buffer or Tris buffer generally used for biochemical reactions, and a weakly alkaline buffer is preferred. If necessary, as mentioned above, the buffer is used to maintain the labeling reaction system of the amino group-containing low molecular weight tumor marker within a suitable pH range.

As described above, for the labeling reaction of the amino group-containing low molecular weight tumor marker, the reaction system may be a liquid phase or liquid phase/solid phase system.

Therefore, the detection kit can also take a corresponding form, the latter system, i.e., a liquid phase/solid phase system is preferred as it maximizes the advantages of this invention. For this purpose, it is preferred that the amino group labeling dye is immobilized on a solid phase. The detection kit having a construction wherein the amino group labeling dye is immobilized on a solid phase, enables measurement of many samples simultaneously, and detection can be easily performed. As already stated, it is particularly preferred that the solid phase used is a microtube or a 96 micro well plate.

The form of the detection kit and container varies with the labeling reaction system (i.e., the combination of the amino group-containing low molecular weight tumor marker and amino group labeling dye), and the kit is not particularly limited if it comprises one set when measuring a sample. One form thereof is given in the examples.

### EXAMPLES

This invention will now be described in more detail, but this invention is not to be construed as being limited in any way thereby.

### Example 1

### Assay of 3-hydroxyproline in Urine Specimen

(Preparation of Immobilized NBD-Cl)

100 μl of 10 mM NBD-Cl acetonitrile solution containing 0.01% of Triton-X and 0.5% of polyethylene glycol were added into the wells of a 96 well plate, and dried in a 30° C. oven for 2 hours.

The solid phase containing the immobilized NBD-Cl (96 well plate) can be stored at a cool and dark place if sealed under a nitrogen atmosphere.

(Pretreatment of Urine Specimen)

Urine sampled from healthy persons (21 samples) and malignant tumor patients (17 samples) was centrifuged at 3000×g for 10 minutes. The supernatant of the urine obtained by centrifugation was collected, and 2 ml of the each supernatant was introduced into a screw-top test tube containing 2 ml of 6M hydrochloric acid, and hydrolyzed at 150° C. for 2 hours. 10 μl of the hydrolyzed urine was lyophilized, and 100 μl of 50 mM borate buffer (pH 10.0) was added thereto.

(Labeling Reaction)

50 μl of the aforesaid treated urine was dispensed into the 96 well plate immobilized with the aforesaid NBD-Cl, and reacted at room temperature for 1 minute with stirring. 100 μl of 0.5M HCl was then added thereto.

(Fluorometry)

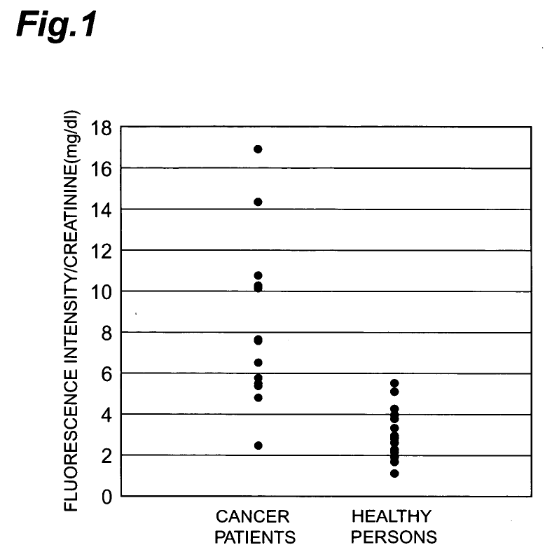

For the sample in each well, the fluorescence intensity was measured using a fluorescence plate reader under the conditions that excitation wavelength is 505 nm and fluorescence wavelength is 560 nm. In order to correct the dilution error based on the water intake (and/or water divergence) of-each person, creatine in the same urine was measured by a creatine test Wako kit (Wako Pure Chemicals), and the 3-hydroxyproline amount in the urine was calculated as a 3-hydroxyproline value per 1 g creatine in the urine (mg/g creatine). FIG. 1 is a plot of thus obtained corrected values.

As shown in FIG. 1, from the fluorometry result of the urine sampled from the malignant tumor patients and the urine extracted from the healthy persons, a difference was observed in the fluorescence intensity value (corrected). Specifically, the measured values for the malignant tumor patients were significantly larger than the measured values for the healthy persons. The average of the healthy persons' measured values was taken, and using this as the threshold value (0.3), the tumor patients' measured values were all larger than the threshold value except for one example (among 17 samples). This test result shows that, apart from one person, the comparison of the fluorescence intensity values measured for all the test subjects, correspond with the clinical diagnosis regarding the presence or absence of malignant tumors in these subjects. Therefore, a malignant tumor can be detected using the malignant tumor detection method and detection kit of the present invention.

Industrial Applicability

As shown above, according to the malignant tumor detection method of the present invention, a malignant tumor can be rapidly and simply detected by calculating the amino group-containing low molecular weight tumor marker amount in the sample, and comparing the calculated value with a threshold value.

Further, the detection of various amino group-containing low molecular weight tumor markers which was previously impossible, can now be performed by means of samples used in general laboratory tests, such as serum and urine.

The malignant tumor detection kit of the present invention is suitable for the application of the method of the present invention, and can be stored for long periods to allow the application of the method of the present invention when required.

According to the preferred examples of the malignant tumor detection method and detection kit of the present invention, the amino labeling dye is immobilized on a solid phase, and as the amino group-containing low molecular weight tumor marker is produced when a sample is added to the solid phase, a malignant tumor can be detected more rapidly. Further, the detection kit can be handled more easily, and permits detection of a large number of samples at the same time.

1. A malignant tumor detection method using an amino group-containing low molecular weight tumor marker in a sample, comprising the steps of:
(a) reacting the sample with an amino group labeling dye to produce a labeled amino group-containing low molecular weight tumor marker;
(b) measuring the fluorescence or absorbance of the sample after the step (a);
(c) calculating the amount of the amino group-containing low molecular weight tumor marker in the sample from the measured value obtained in the step (b); and
(d) comparing the calculated value obtained in the step (c) with the threshold value for the amino group-containing low molecular weight tumor marker, where the result is deemed to be “positive” for the presence of a malignant tumor if the calculated value is larger than the threshold.

2. The malignant tumor detection method according to claim 1, wherein said amino group labeling dye is immobilized on a solid phase in advance.

3. The malignant tumor detection method according to claim 1, further comprising a step of pretreating the sample prior to the step (a).

4. The malignant tumor detection method according to claim 3, further comprising a step of performing a concentration correction of the calculated value of the amino group-containing low molecular weight tumor marker between the steps (c) and (d)

5. The malignant tumor detection method according to claim 4, wherein the amino group-containing low molecular weight tumor marker is 3-hydroxyproline.

6. The malignant tumor detection method according to claim 5, wherein the amino group labeling dye is 4-chloro-7-nitrobenzofurazan or 4-fluoro-7-nitrobenzofurazan.

7. The malignant tumor detection method according to claim 6, wherein the step (b) is performed by fluorometry.

8. The malignant tumor detection method according to claim 7, wherein the amino group labeling dye is immobilized on a solid phase in advance.

9. The malignant tumor detection method according to claim 2, wherein the sample is urine.

10. The malignant tumor detection method according to claim 9, further comprising a step of performing a concentration correction of the calculated value of the amino group-containing low molecular weight tumor marker between the steps (c) and (d).

11. The malignant tumor detection method according to claim 10, wherein the concentration correction is performed by the creatinine amount in the sample.

12. A malignant tumor detection kit using an amino group-containing low molecular weight tumor marker in a sample, comprising at least a standard solution of the amino group-containing low molecular weight tumor marker, an amino group labeling dye and a buffer.

13. The malignant tumor detection kit according to claim 12, wherein said amino group labeling dye is immobilized on a solid phase.
